(19) United States
(12) Patent Application Publication
(10) Pub. No.: US 2010/0092395 A1
(43) Pub. Date: Apr. 15, 2010

(54) ACOUSTICALLY DELIVERING METHODS AND COMPOSITONS FOR REMOTE TREATMENT OF A TUMOR

(75) Inventors: Lih-Hwa HWANG, Taipei (TW); Wen-Shiang CHEN, Taipei (TW)

Correspondence Address: BRIAN M. MCINNIS, 12th Floor, Ruttonjee House, 11 Duddell Street, Hong Kong, HK

(73) Assignee: NATIONAL TAIWAN UNIVERSITY, Taipei (TW)

(21) Appl. No.: 12/541,965

(22) Filed: Aug. 16, 2009

Related U.S. Application Data
(60) Provisional application No. 61/104,869, filed on Oct. 13, 2008.

(51) Int. Cl. A61K 49/22; A61K 49/00

(52) U.S. Cl. 424/9.52; 424/9.1

(57) ABSTRACT

Disclosed herein is a method of acoustically delivering a therapeutic composition to a subject pre-diagnosed with a tumor. The method comprises the steps of: parenterally administering the composition from a site remote from a tumor location; and exposing the parenterally administering site to ultrasound waves to target the delivery of the composition. The therapeutic composition comprises an effective amount of a polypeptide or a plasmid nucleic acid encoding the polypeptide, the polypeptide or the nucleic acid is suspended in a dispersed medium; and an effective amount of an microbubble contrast agent; wherein the polypeptide is an angiogenesis inhibitor, and the therapeutic composition is capable of reducing the size of the tumor without the risk of inducing viral vector-induced immunogenicity in the subject.

### CROSS REFERENCES

This application claims the benefit of U.S. provisional application Ser. No. 61/104,869, filed on Oct. 13, 2008.

### BACKGROUND

1. Field of Disclosure

The present disclosure in general relates to methods and compositions for treating a subject pre-diagnosed with a tumor. Specifically, this disclosure relates to acoustically deliver a therapeutic composition comprising a nucleic acid of an angiogenesis inhibitor and a microbubble contrast agent to a subject with hepatic tumor.

2. Description of Related Art

Gene therapy refers to procedures in which therapeutic genes are delivered to target cells in a subject with conditions that may benefit from the therapeutic genes. The efficient transfer and expression of the therapeutic genes in cells is accomplished by inserting them into vectors. The function of the vectors is to protect the therapeutic genes and to transport them safely into the nuclei of the target cells, where they can finally be decoded (i.e., expressed) to produce a therapeutic protein. Vectors are classified into viral and non-viral vectors. Gene delivery by non-viral vectors is accomplished by uptaking therapeutic genes condensing with lipids, peptides, proteins, inactivated virus particles or crystals of calcium phosphate into cells; or by coating therapeutic genes onto microprojectiles and fire them into the nuclei of target cells by a gene gun. However, efficiency of gene transfer by non-viral vectors is usually too low to give real benefit. Viral vectors, on the other hand, generally produce a much higher efficiencies of gene delivery than non-viral vectors. Viral vectors are constructed by removing viral genes to allow insertion of therapeutic genes. In principle, any virus can provide basis for a vector, but retroviral and adenoviral vectors have been the most widely used. However, several problems of viral gene therapy must be overcome before it gains widespread use. Viral vector-induced immunogenicity (i.e., immuno response to viral vectors) not only impedes the delivery of genes to target cells but also causes severe complications to the patient. In one of the early gene therapy trials in 1999 this led to the death of the patient, who was treated using an adenoviral vector (Beardsley T, February 2000, “A tragic death clouds the future of an innovative treatment method.” Scientific American). Other viral vectors, such as lentiviruses, insert their genomes at a seemingly random location on one of the host chromosomes can disturb the function of cellular genes and lead to cancer. In a severe combined immunodeficiency retroviral gene therapy trial conducted in 2002, two of the patients developed leukemia as a consequence of the treatment (McDowell N, 15 Jan. 2003, “New cancer case halts US gene therapy trials.” New Scientist).

Hence, there remains a need for developing successful strategies for delivering therapeutic genes into target cells, and strategies for repeated administration of therapeutic genes for optimal therapeutic benefits without the risk of causing vector-induced immunogenicity in the patient.

### SUMMARY

The present disclosure is based on the unexpected finding that ultrasound waves may enhance the delivery of a naked plasmid nucleic acid, particularly a nucleic acid with anti-angionenesis function loaded in a plasmid and not in a viral vector such as an adenoviral vector, the plasmid nucleic acid is suspended in a saline or a buffer solution and intramuscularly injected from a site remote from where hepatocellular carcinoma (HCC) is located along with a microbubble contrast agent, and thereby reducing the size of HCC without causing an immune response commonly associated with the adenoviral vectors in the subject.

Accordingly, it is therefore an object of the present disclosure to provide a method of acoustically enhanced delivering a therapeutic composition to a subject pre-diagnosed with a tumor. The method includes steps of:

(a) parenterally administering the therapeutic composition intramuscularly from a site remote from a tumor location, the therapeutic composition comprising:
an effective amount of at least one polypeptide or at least one plasmid nucleic acid encoding the polypeptide, the polypeptide or the plasmid nucleic acid is suspended in a dispersion medium; and
an effective amount of a microbubble contrast agent; and

(b) exposing the parenterally administering site of step (a) to ultrasound waves to enhance the delivery of the therapeutic composition;

wherein the polypeptide is an angiogenesis inhibitor, and the therapeutic composition is capable of reducing a size of the tumor.

The angiogenesis inhibitor is selected from the group consisting of IFN-α, IFN-β, IFN-γ, IL-4, IL-8, IL-12, IL-18, platelet factor-4, angiopoietin 2, angiostatin, endostatin (ED), platelet factor-4, osteopontin, maspin, canstatin, proliferin-related protein, prolactin and calreticulin (CRT). In one example, the angiogenesis inhibitor is ED. In another example, the angiogenesis inhibitor is CRT. The tumor can be any of pancreatic tumor, lung cancer, colon cancer, gastric cancer, breast cancer, prostate cancer, hepatocellular carcinoma, melanoma, glioblastoma, brain tumor, hematopoietic malignancies, retinoblastoma, renal cell carcinoma, head and neck cancer, cervical cancer, esophageal cancer, and squamous cell carcinoma. In one preferred example, the tumor is a hepatocellular carcinoma. The parenterally administering step includes intramuscularly injection. The dispersion medium is any of water, a buffer solution, an isotonic sodium chloride solution, oils, or fatty acids. The microbubble contrast agent is composed of a shell and a gas core, wherein the shell is formed by a material selected from the group consisting of albumin, galactose, lipid, polymer and combinations thereof; and the gas core is formed by any of air, octafluoropropane, perfluorocarbon, sulfur hexafluoride or nitrogen. In one example, the microbubble contrast agent has a lipid shell and a gas core formed by sulfur hexafluoride, and an averaged diameter of about 2.5 μm.

In one preferred example, the nucleic acid and the microbubble contrast agent are mixed in a ratio of 7:3 (v/v). The ultrasound waves have an intensity of about 0.5˜4 W/cm² and are administrated for about 1˜30 min. In one preferred example, the ultrasound waves are administered in an intensity of about 2 W/cm² for about 10 min. The composition is administered either intermittently or consecutively to the subject. In one example, the composition is administered intermittently, i.e., at least every 2˜7 days with a dose of 10 μg˜10 mg plasmid nucleic acid/Kg body weight or 1˜100 mg polypeptide/Kg of body weight for at least 4 times. In another example, the composition is administered consecutively, i.e., every day with a dose of 10 μg˜10 mg plasmid nucleic acid/Kg body weight or 1˜100 mg polypeptide/Kg of body weight for at least 4 days. In one aspect, the method may further comprise the step of administering to the subject a chemotherapeutic agent before, at the same time or after administering the therapeutic composition of this disclosure. The chemotherapeutic agent is selected from the group consisting of cisplatin, carboplatin, oxaliplatin, azathioprine, mercaptopurine, vincristine, vinblastine, vinorelbine, vindesine, paclitaxel, docetaxel, camptothecins, irinotecan, topotecan, amsacrine, etoposide, etoposide phosphate, teniposide, dactinomycin, trastuzumab and cetuximab, rituximab. In another aspect, the method further comprises the step of administering to the subject an adenoviral vector of a nucleic acid encoding a polypeptide selecting from the group consisting of GM-CSF, IL-12, ED and PEDF, before, at the same time or after the therapeutic composition of this disclosure is administered. In one preferred example, the adenoviral vector is administered intratumorally to the subject before the therapeutic composition of this disclosure is administered.

It is another object of the present disclosure to provide a therapeutic composition delivered acoustically to a subject for treating a tumor, comprising: an effective amount of a polypeptide or a plasmid nucleic acid encoding the polypeptide, in which the polypeptide or the plasmid nucleic acid is suspended in a dispersion medium; and an effective amount of a microbubble contrast agent; wherein the polypeptide is an angiogenesis inhibitor, and the therapeutic composition is capable of reducing a size of the tumor to at least half of the control. In one preferred example, the angiogenesis inhibitor is ED or CRT, the microbubble contrast agent is SONOVUE®, the dispersion medium is a buffer solution and the nucleic acid and the microbubble contrast agent are mixed in a ratio of 7:3 (v/v). The therapeutic composition is administered to the subject either intermittently or consecutively to a subject in need of such treatment in accordance with one method of this disclosure. In another aspect, the composition is administered to the subject before, at the same time or after administration of a chemotherapeutic agent. In another example, the composition is administered to the subject before, at the same time or after administration of an adenoviral vector comprising a nucleic acid encoding a polypeptide selecting from the group consisting of GM-CSF, IL-12, ED and PEDF. The adenoviral vector is administered intratumorally to the subject before the composition is injected.

In conclusion, the disclosure allows reduction of a size of a solid tumor to at least half of the control by intramuscularly injecting a plasmid nucleic acid of an angiogenesis inhibitor to a subject pre-diagnosed with the tumor from a site remote from the tumor location with an aid of ultrasound waves. Preferably, the therapeutic nucleic acid of this disclosure is loaded in a plasmid vector, hence, eliminating the risk of developing viral vector-induced immunogenicity in the subject receiving such vector, which oftentimes limits the application of the therapeutic nucleic acid. The composition and/or method of this disclosure also overcome the disadvantages of the prior art composition and/or method by intramuscularly injected the composition from a site remote from the tumor location (e.g., any site on the four limbs), instead of delivering intratumorally as commonly seen in viral gene therapy. This makes the method and/or composition of this disclosure more easy to use, for most of the tumors are embedded inside the body and are not easily accessed. Furthermore, the composition and/or method of this disclosure can be repeatedly administered to the subject for several times (i.e., at least 4 times as demonstrated in the Examples) to achieve optimal therapeutic benefits, i.e., a reduction of at least 50% of the tumor size.

These and other features, aspects, and advantages of the present disclosure will become better understood with reference to the following description and appended claims.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure. In the drawings,

FIG. 1 illustrates the effect of (A) intermittent or (B) consecutive ultrasound therapy performed intramuscularly on mice with subcutaneous hepatic tumor in accordance with one embodiment of this disclosure, the arrow indicates the day when the tumor cells were inoculated or when the respective ultrasound therapy was given to the animal;

FIG. 2 illustrates the tumor weight measured on day 28 for mice receiving (A) intermittent or (B) consecutive ultrasound therapy performed intramuscularly on mice with subcutaneous hepatic tumor in accordance with one embodiment of this disclosure, the arrow indicates the day when the tumor cells were inoculated or when the respective ultrasound therapy was given to the animal, *p<0.05, **p<0.005, ***p<0.001;

FIG. 3 illustrates the tumor weight measured on day 28 for mice receiving (A) intermittent or (B) consecutive ultrasound therapy performed intramuscularly on mice with orthotopic hepatic tumor in accordance with one embodiment of this disclosure, the arrow indicates the day when the tumor cells were inoculated or when the respective ultrasound therapy was given to the animal, *p<0.05, **p<0.005;

FIG. 4 are survival curves for mice receiving intermittent ultrasound therapy for orthotopic hepatic tumor in accordance with one embodiment of this disclosure, the arrow indicates the day when the tumor cells were inoculated or when the respective ultrasound therapy was given to the animal;

FIG. 5A illustrates the tumor weight measured on day 14 for rats receiving intermittent (i.e., every 3 days) ultrasound therapy for multifocal hepatic tumors in accordance with one embodiment of this disclosure;

FIG. 5B illustrates the tumor weight measured on day 14 for rats receiving consecutive ultrasound therapy (i.e., once every day for 4 days in sequence) for multifocal hepatic tumors in accordance with one embodiment of this disclosure, the arrow indicates the day when the respective ultrasound therapy was given to the animal, *p<0.05, **p<0.005, ***p<0.001;

FIG. 6 illustrates the tumor burdens measured on (A) day 40 or (B) day 27 for mice or rats, respectively receiving combined immunotherapy (Ad/G+I) and ultrasound therapy at indicated times (represented by the arrows) for orthotopic hepatic tumors in accordance with one embodiment of this disclosure, *p<0.05, **p<0.005, ***p<0.001;

FIG. 7 illustrate the immunohistochemical staining of CD31+ cells measured on (A) day 28 for mice receiving intermittent ultrasound therapy only or (B) day 40 for mice receiving combined immunotherapy (Ad/G+I) and intermittent ultrasound therapy at indicated times (represented by the arrows), ***p<0.001;

FIG. 8 illustrate the immunohistochemical staining of CD4+ cells measured on (A) day 28 for mice receiving intermittent ultrasound therapy only or (B) day 40 for mice receiving combined immunotherapy (Ad/G+I) and intermittent ultrasound therapy at indicated times (represented by the arrows), ***p<0.001, *p<0.05, **p<0.005, and ***p<0.001; and

FIG. 9 illustrate the immunohistochemical staining of CD8 cells measured on (A) day 28 for mice receiving intermittent ultrasound therapy only or (B) day 40 for mice receiving combined immunotherapy (Ad/G+I) and intermittent ultrasound therapy at indicated times (represented by the arrows), ***p<0.001.

### DESCRIPTION OF THE PREFERRED EMBODIMENTS

Described herein are methods and compositions for treating a subject pre-diagnosed with a tumor by use of a plasmid nucleic acid of an angiogenesis inhibitor suspended in a suitable medium and injected intramuscularly from a site remote from the tumor location with an aid of ultrasound waves.

Ultrasound waves have been used to image the human body for at least half a century; it is one of the most widely used diagnostic tools in modern medicine. Contrast enhanced ultrasound is the application of ultrasound contrast agent to traditional sonography. Ultrasound contrast agents are gas-filled microbubbles that are usually administered intravenously to the systemic circulation to reflect the ultrasound waves and produce a unique sonogram with increased contrast due to echogenicity differences between the gas in the microbubbles and the soft tissues surrounding the body. Thus, ultrasound contrast agents traditionally are used to image blood perfusion in organs, measure blood flows in the heart and etc. Recently, microbubbles are formulated to carry therapeutic agents as well. Hydrophilic compounds can be encased within lipid membranes or polymeric shells that stabilize the microbubbles. Albumin-coated microbubble has been proposed to be an effective means for delivering a reporter gene comprised within an adenovirus gene vector to a mouse heart (Shohet et al., “Echocardiographic destruction of albumin microbubbles directs gene delivery to the myocardium.” Circulation 2000 101:2554-2556). Inventors of this study take advantage of the ultrasound-mediated microbubbles disruption as a delivery means for in vivo targeting a therapeutic gene, particularly the gene of an angiogenesis inhibitor, the gene is loaded into a plasmid instead of being comprised in a viral vector, and suspended in a saline or a buffer solution and acoustically deliver to a subject pre-diagnosed with a tumor, thereby successfully eliminating the vector-induced immunogenecity problem commonly associated with viral gene therapy, and the therapeutic gene of the improved method of this disclosure may be administered several times, instead of just once as in the known viral gene therapy, to achieve optimal therapeutic benefits. Any suitable plasmid vector may be used to practice this disclosure, and the selection of plasmids may be easily obtained by any skilled person in the related art without undue experimentation.

It is therefore features of this disclosure to provide methods and compositions for treating a subject pre-diagnosed with a tumor.

In one aspect, this disclosure provides a method of treating a subject pre-diagnosed with a tumor, by injecting a therapeutic composition intramuscularly from a site remote from a location of the tumor, and exposing the injecting site to ultrasound waves to enhance the delivery of the therapeutic composition. The therapeutic composition comprises an effective amount of at least one polypeptide or at least one plasmid nucleic acid encoding the polypeptide, the polypeptide or the plasmid nucleic acid is suspended in a dispersion medium; and an effective amount of a microbubble contrast agent, wherein the polypeptide is an angiogenesis inhibitor, and the composition is capable of reducing a size of the tumor, to at least half of the control.

The angiogenesis inhibitor is a substance that inhibits angiogenesis, i.e., the growth of new blood vessels. Every solid tumor needs to generate blood vessels to keep it alive once it reaches a certain size. Usually, blood vessels are not built elsewhere in an adult body unless tissue repair is actively in process. The angiostatic agent (such as ED) can suppress the building of blood vessels, preventing the cancer from growing indefinitely. Suitable selection of angiogenesis inhibitor in this disclosure include, but are not limited to, IFN-α, IFN-β, IFN-γ, IL-4, IL-8, IL-12, IL-18, platelet factor-4, angiopoietin 2, angiostatin, endostatin (ED), platelet factor-4 osteopontin, maspin, canstatin, proliferin-related protein, prolactin and calreticulin (CRT). In one example, the angiogenesis inhibitor is ED. In another example, angiogenesis inhibitor is CRT.

There are a variety of microbubbles contrast agents. Microbubble is composed of a shell and a gas core. Selection of shell material determines how easily the microbubble is taken up by the immune system. A more hydrophilic material tends to be taken up more easily, which reduces the microbubble residence time in the circulation, and thereby reducing the time available for contrast imaging. The shell material also affects microbubble mechanical elasticity. The more elastic the material, the more acoustic energy it can withstand before bursting. Currently, microbubble shells are composed of albumin, galactose, lipid or polymers. The gas core is the most important part of the ultrasound contrast microbubble because it determines the echogenecity. When gas bubbles are caught in an ultrasound frequency field, they compress, oscillate, and reflect a characteristic echo, this generates the strong and unique sonogram in contrast-enhanced ultrasound. Gas cores can be composed of air, or heavy gases like octafluoropropane, perfluorocarbon, sulfur hexafluoride or nitrogen. Heavy gases are less water-soluble so they are less likely to leak out from the microbubble to impair echogenecity. In general, each of the microbubbles has a diameter of about 2 to 4 μm in average, such as 2, 2.5, 3, 3.5 or 4 μm. OPTISON® (made by GE healthcare) is the first microbubble approved by Food and Drug Administration (FDA), and has an albumin shell and octafluoropropane (C₃F₈) gas core. The second FDA-approved microbubble, LEVOVIST®, (made by Schering AG), has a palmitic acid/galactose shell and an air core. Other examples of microbubble include, but are not limited to ALBUNEX®) (made by Molecular Biosystems), SONOVUE® (made by Bracco Diagnostics, Inc.), SONOZOID® (made by Schering AG), SONOVIST® (made by Schering AG), and DEFINITY® (made by DuPont Pharmaceuticals). ALBUNEX® has an albumin shell and an air core. SONOVUE®& contains a sulfur hexafluoride (SF₆) gas core that is stabilized in aqueous dispersion of a monolayer of phospholipids. SONOZOID® is another microbubble preparation containing a perfluorocarbon gas core and a lipid shell. DEFINITY® is another FDA-approved microbubble that contains a lipid shell and an octafluoropropane (C₃F₈) gas core. In one preferred example, the microbubble contrast agent is SONOVUE®.

It is another aspect of this disclosure to provide a therapeutic composition for treating a tumor. The composition is prepared by mixing a plasmid nucleic acid encoding an angiogenesis inhibitor with the microbubble contrast agent (such as SonoVue®, prepared in accordance with the manufacturer's instruction) in a ratio between about 9:1 to 1:9 (v/v), preferably between about 4:1 to 1:4 (v/v), and most preferably in a ratio of about 7:3 (v/v). The plasmid nucleic acid is suspended in a suitable dispersion medium, such as water, PBS, saline, oils, or fatty acids. The therapeutic compositions thus prepared may be administered parenterally, by inhalation spray, topically, rectally, nasally, buccally or vaginally. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the composition is administered intramuscularly, intraperitoneally or intravenously, and most preferably, the composition is administered intramuscularly. In one example, the composition of this disclosure is injected intramuscularly from a site on one limb (i.e., arm or leg) of the subject. The body portion suitable for injection is selected based on the followings, such as the choice of the plasmid nucleic acid or the polypeptide to be released, the subject's personal condition including sex, age, body weight, and/or current and prior medical conditions. An experienced physician may determine suitable body portion for injection without undue experiment. In one example, the body portion is an upper arm region of a human. Sterile injectable forms of the composition of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, phosphate buffer solution and isotonic sodium chloride solution (i.e., saline). In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The dosage required depends on the choice of the route of administration; the nature of the formulation; the nature of the subject's illness; the subject's weight, surface area, age and sex; other drugs being administered; and the judgment of the attending physician. Suitable dosages are from 10 μg˜10 mg plasmid nucleic acid/Kg of body weight or 1˜100 mg polypeptide/Kg of body weight. Variations in the needed dosage are to be expected in view of the variety of angiogenesis inhibitors available and the different efficiencies of various routes for administration. Those of skill in the art can readily evaluate relevant factors and based on this information, determine the particular dosage to be used for an intended purpose.

The injected site of the subject is subsequently exposed to therapeutic ultrasound waves with an intensity between 0.5˜4 W/cm² for about 1˜30 min. In one preferred example, the injected site is exposed to ultrasound waves with an intensity of about 2 W/cm² for about 10 min. It is believe that exposure to ultrasound waves help target the therapeutic composition of this disclosure to the tumor location and thereby achieving therapeutic effects (i.e., reduction the size or volume of the tumor) by expressing the encoded angiostatic agent to suppress blood vessel building process in the tumor region.

The disclosure features methods and compositions for treating a subject pre-diagnosed with a tumor or a cancer. A subject herein refers to a human and a non-human animal. Examples of a non-human animal include all vertebrates, e.g., mammals, such as primates, dogs, rodents (e.g., mouse or rat), cats, sheep, horses or pigs; and non-mammals, such as birds, amphibians, reptiles and etc. In a preferred example, the subject is a human. Examples of the tumor or cancer include pancreatic tumor, lung tumor, colon cancer, gastric cancer, breast cancer, prostate cancer, hepatocellular carcinoma, melanoma, glioblastoma, brain tumor, hematopoietic malignancies, retinoblastoma, renal cell carcinoma, head and neck cancer, cervical cancer, esophageal cancer, and squamous cell carcinoma. In one example, the subject has been diagnosed with a hepatic tumor. The subject may have received medical treatment before being subjected to the method and/or composition of this disclosure. The medical treatment herein refers to surgery or radiotherapy commonly applied to a patient with tumor. Inventors of this study have reported previously the effectiveness of a recombinant adenovirus carrying at least one gene selected form the group consisting of granulocyte-macrophage colony-stimulating factor (GM-CSF), interleukin-12 (IL-12), endostatin (ED), and pigment epithelium-derived factor (PEDF) on mice and/or rat HCC model, see U.S. 60/957,865, filed on Aug. 24, 2007 and U.S. Ser. No. 12/229,389 filed on Aug. 22, 2008, the disclosure of the two US applications are incorporated herein by reference. Therefore, to augment the anti-tumor effects of gene therapy, the subject pre-diagnosed with tumor may also receive adenoviral gene therapy before, at the same time or after subjecting to methods and/or compositions of this disclosure. In one example, the subject is treated with adenoviral gene therapy as described above before the method and/or composition of this disclosure is conducted. The therapeutic genes encoded within the adenoviral vectors are at least one gene selected from the group consisting of GM-CSF, IL-12, ED and PEDF.

Also within the scope of this disclosure is the combinational use of the therapeutic composition of this disclosure with a chemotherapeutic agent to enhance anti-tumor effects. The chemotherapeutic agent may be administered before, at the same time or after administering the composition of this disclosure. The chemotherapeutic agent is selected from the group consisting of cisplatin, carboplatin, oxaliplatin, azathioprine, mercaptopurine, vincristine, vinblastine, vinorelbine, vindesine, paclitaxel, docetaxel, camptothecins, irinotecan, topotecan, amsacrine, etoposide, etoposide phosphate, teniposide, dactinomycin, trastuzumab, cetuximab, and rituximab.

All other acronyms and abbreviations have the corresponding meaning as published in journals related to the arts of chemistry and biology.

Other than in the operating examples, or where otherwise indicated, all numbers expressing quantities of ingredients, reaction conditions, and so forth used in this application are to be understood as being modified in all instances by the term “about.” Accordingly, unless the contrary is indicated, the numerical parameters set forth in this application are approximations that may vary depending upon the desired properties sought to be obtained by the present disclosure.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in the respective testing measurements.

Reference will now be made in detail to the present preferred embodiments of the disclosure, examples of which are illustrated in the accompanying drawings. Wherever possible, the same reference numbers are used in the drawings and the description to refer to the same or like parts.

### EXAMPLES

### Materials and Methods

### Cell Lines and Animals

The mouse hepatoma cell line BNL and human embryonic kidney cell line 293 were purchased from the American Type Culture Collection (Rockville, Md.). Both cell lines were maintained in Dulbecco's modified Eagle's medium (DMEM; Seromed, Berlin, Germany) supplemented with 10% fetal calf serum (FCS; Biological Industries, Israel). Male BALB/c mice aged 7-8 weeks and male Wistar rats aged 6-7 weeks were used in these experiments. All animal experiments were performed in accordance with the guidelines of the Animal Welfare Committee of National Taiwan University College of Medicine.

### Preparation of Plasmid DNA

The expression plasmid encoding an endostatin (ED) or a calreticulin (CRT) was constructed using the pcDNA3 vector (Invitrogen, San Diego, Calif.). The ED or CRT cDNA was cloned downstream of the cytomegalovirus (CMV) immediate early gene promoter. Both expression plasmids were amplified by E. coli culture and purified using the Giga kit of QIAGEN EndoFree™.

### Construction of Adenoviral Vectors

The adenoviral vector containing a mouse GM-CSF cDNA (Ad/GM-CSF), IL-12 cDNA (Ad/IL-12), ED cDNA (Ad/ED), PEDF cDNA (Ad/PEDF) or an enhanced green fluorescence protein (EGFP) gene (Ad/GFP) under the control of a CMV immediate early gene promoter was constructed using the AdEasy system (He T C et al. Proc Natl Acad Sci USA 1998; 95:2509-2514) as previously described (Tai K F et al. J Gene Med 2003; 5:386-398). Ad/IL-12, kindly provide by Dr. B. L. Chiang of National Taiwan University College of Medicine, is the adenoviral vector containing a murine single-chain IL-12 gene that encodes the two IL-12 subunits (p35 and p40) linked by a polypeptide linker (Lee Y L et al. Hum Gene Ther 2001; 12:2065-2079). The recombinant adenovirus was propagated in human embryonic kidney 293 cells. Infected cells were lysed after infection, and virus particles were purified and stored in frozen condition until used.

Generation of Orthotopic Liver Tumors and in vivo Gene Therapy

For inoculated tumor model, 3×10⁵ BNL cells were injected subcutaneously to generate s.c. tumors or injected into the left liver lobe to generate orthotopic liver tumors (5×10⁵ cells per injection) at day 1. The needle hole was sealed with an electric coagulator (Aaron, Petersburg, Fla., USA) immediately after the withdrawal of the needle to avoid leakage of the injected substance. The incision was subsequently sutured.

For ultrasound therapy, animals were intramuscularly injected with 30 μl of a plasmid DNA solution each time, followed by exposing the injecting site to ultrasound waves (intensity: 2 W/cm²; frequency: 1 MHz; duty cycle: 20%) for 10 min at the indicated time. The plasmid DNA solution was prepared by withdrawing 10 μl of SONOVUE® (Bracco Diagnostics, Inc. USA) from its container and added to 20 μl saline solution containing either 100 μg of ED- or CRT-plasmid DNA or empty plasmid vector, and mixed thoroughly. The final concentration of SONOVUE® in the injecting solution is 33% (v/v). Tumor sizes were measured using calipers. Tumor volume was calculated using the formula: volume=width²×length×0.52. In the case of a combination therapy, a single injection of 30 μl of adenoviruses, 1×10⁹ Ad/LacZ, or 0.5×10⁹ Ad/GM-CSF+0.5×10⁹ Ad/IL-12 (i.e., Ad/G+I) was first administered intratumorally on day 14 (n=5 for each group) after tumor implantation, followed by ultrasound therapy at the indicated time using the same conditions as described above.

For primary multifocal HCC model, Wistar rats were given diethyinitrosamine (DEN) solution daily for 6 weeks, starting with 100 ppm for the first week. The average body weight (BW) of the animals was measured weekly per group of five rats, and the concentration of DEN in their drinking water was adjusted proportionally to the BW at each week relative to that of the first week. After 6 weeks of DEN administration, the animals were given regular water for another 6 weeks to allow tumor progression. For ultrasound therapy, animals were injected with a plasmid DNA solution each time intramuscularly, followed by exposure to ultrasound at the indicated time using the same conditions as described above. The plasmid DNA solution was prepared in accordance with the procedures described above, except 200 μg plasmid DNA (empty plasmid vector, ED-, or CRT-expressing plasmid DNA) was used instead of 100 μg plasmid DNA. In the case of a combination therapy, adenoviruses (same doses as described above) in 100 μl were first injected through portal vein into the liver, followed by ultrasound therapy at the indicated time using the same conditions as described above. After treatment, rats were sacrificed and all liver lobes were promptly harvested, weighed, and the diameters of all of the macroscopically visible nodules on the liver surface and in the 5-mm sliced sections were measured. Tumor burdens were determined by the total volume of all the tumor nodules with diameter greater than 3 mm.

### Immunohistochemistry Staining

Tumor tissues were removed from the animals and imbedded in Tissue-Tek OCT compound, snap-frozen in liquid nitrogen, and stored at −80° C. for further use. Five-micron cryosections were sliced using a model CM1900 cryostat (Leica, Bannockburn, Ill.), air-dried, and fixed with acetone/chloroform mixture (1:1) at −20° C. for 10 minutes. Specimens were treated with 0.03% of hydrogen peroxide in PBS for 10 minutes to block endogenous peroxidase activity. The cryosections sections were then probed with monoclonal rat-anti-mouse CD31 (clone MEC13.3, BD PharMingen™, San Diego, Calif., USA) at a concentration of 2.5 μg/ml for 2 hours at room temperature. For the immunohistochemistry staining of mouse CD4+ T cells and CD8+ T cells, the sections were probed with monoclonal rat anti-mouse CD4 (Clone H129.19, BD PharMingen™, San Diego, Calif., USA) and monoclonal rat anti-mouse CD8a (Clone 53-6.7, BD PharMingen™, San Diego, Calif., USA), respectively. The sections were washed for a few times followed by treatment with biotinylated rabbit-anti-rat immunoglobulin antibody (Dako, Carprinteria, Calif., USA) for 1 hour at room temperature. The sections were washed again and developed using a Dako Envision+ system-HRP (DAB) (Dako, Carprinteria, Calif., USA) kit according to the manufacturer's instructions.

### Results

### Acoustically Delivery of ED or CRT Plasmid DNA Reduces Orthotopic Liver Tumors in Mice

Mice were inoculated with liver tumor cells in accordance with the procedures described above. Usually, a tumor nodule of 10˜20 mm³ and 60˜100 mm³ could be observed on day 7 and day 14, respectively, after tumor implantation, and each representing an intermediate tumor burden and a large tumor burden, respectively. Two different ultrasound therapy protocols (intermittent vs consecutive) were adopted in this study for targeting the injecting DNA expression in vivo for the treatment of mice with subcutaneous hepatic tumor. In intermittent protocol, the steps of intramuscularly injecting a plasmid DNA solution and ultrasound exposure were repeated every 7 days for at least 4 times, whereas in consecutive protocol, the entire procedures were repeated every day for at least 4 days.

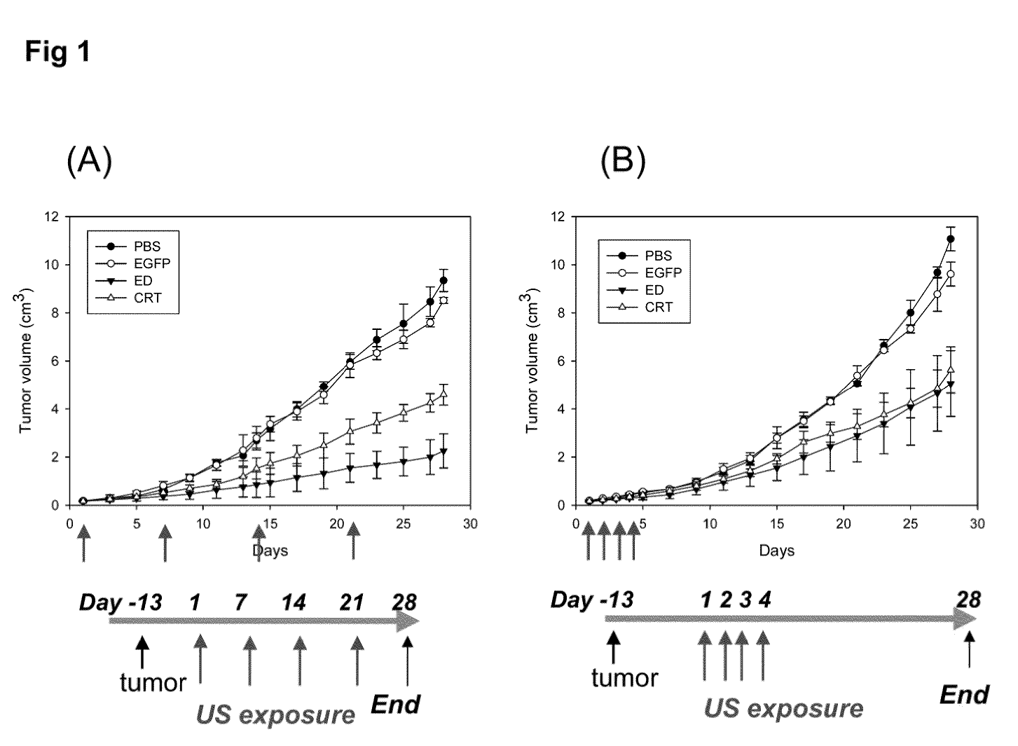

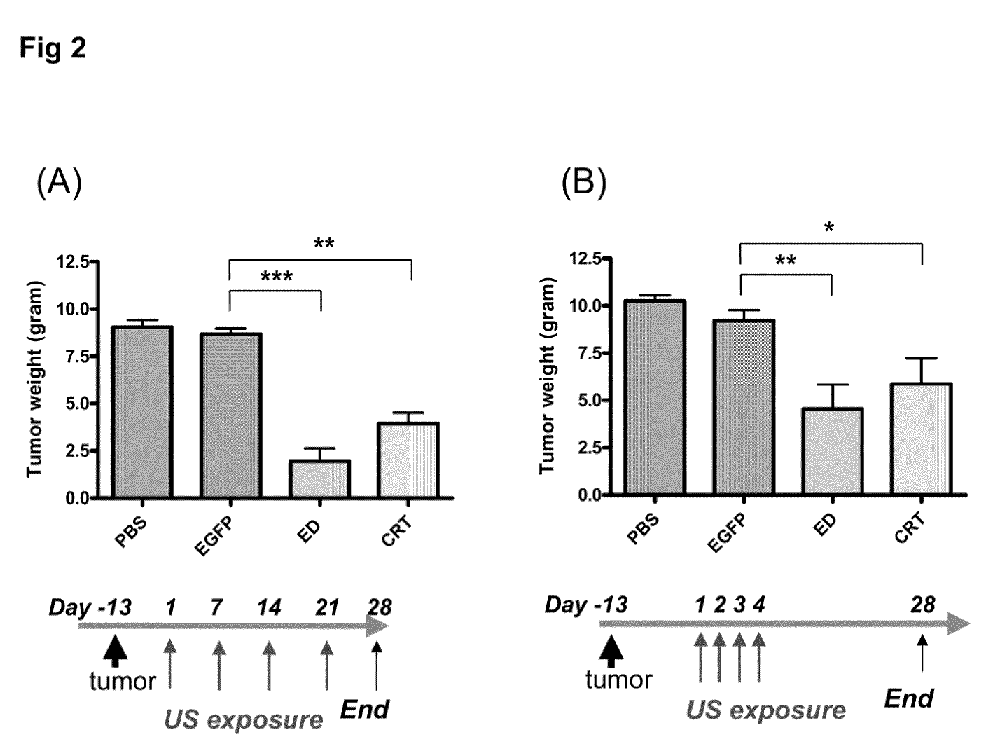

FIG. 1A illustrates the effects of ultrasound therapy applied intermittently on days 1, 7, 14 and 21, respectively on mice with subcutaneous hepatic tumor. After intermittent treatment with either ED or CRT plasmid DNA for 4 times, tumor size shrank to ¼ and ½ of the control animals, respectively. The results were further confirmed by sacrificing the animals and measuring the tumor weight at the end of the experiment, i.e., on day 28. The average weight of the tumor for mice receiving ultrasound ED treatment and ultrasound CRT treatment is about 2/9 and 4/9 of that of the control animals, respectively (FIG. 2A).

FIG. 1B illustrates the effect of consecutive ultrasound therapy applied consecutively on days 1, 2, 3 and 4, respectively on mice with subcutaneous hepatic tumor. FIG. 2B illustrated the tumor weight measure on day 28 for mice receiving consecutive ultrasound therapy. As the finding provided in intermittent therapy, tumor volume, as well as tumor weight, are both significantly smaller for the mice receiving ultrasound ED or CRT treatment.

It also appears that ED was more effective than CRT in regressing the growth of tumor, with the tumor size and weight measured on day 28 being much smaller than that with CRT treatment (FIGS. 1 and 2); and ultrasound therapy delivered intermittently was more effective than that delivered consecutively (FIG. 1).

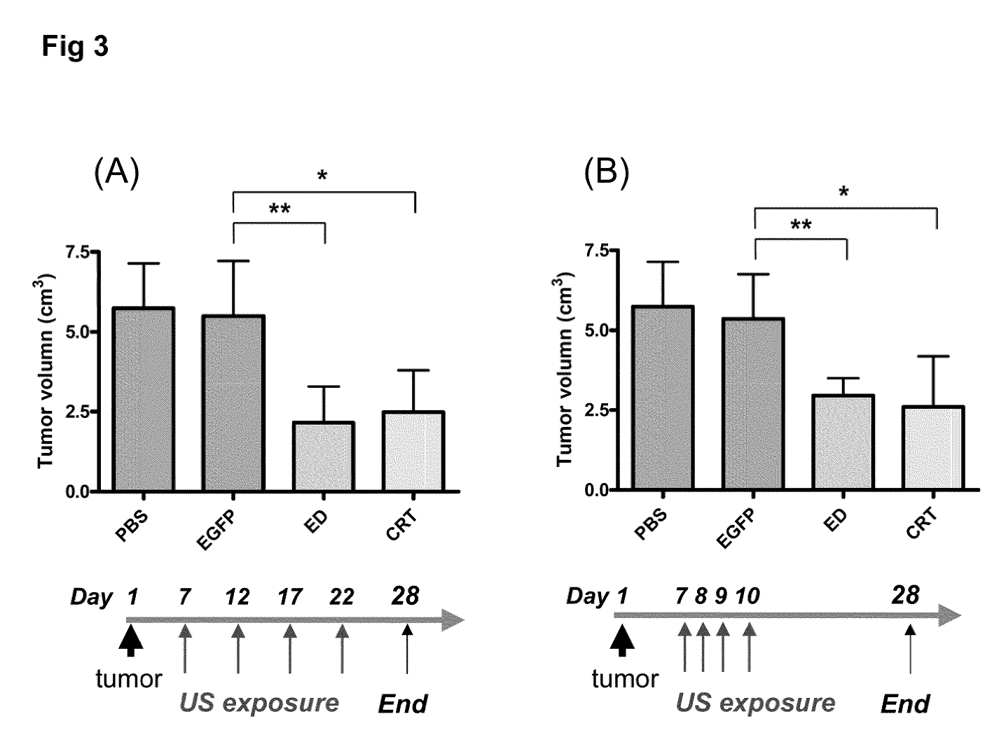

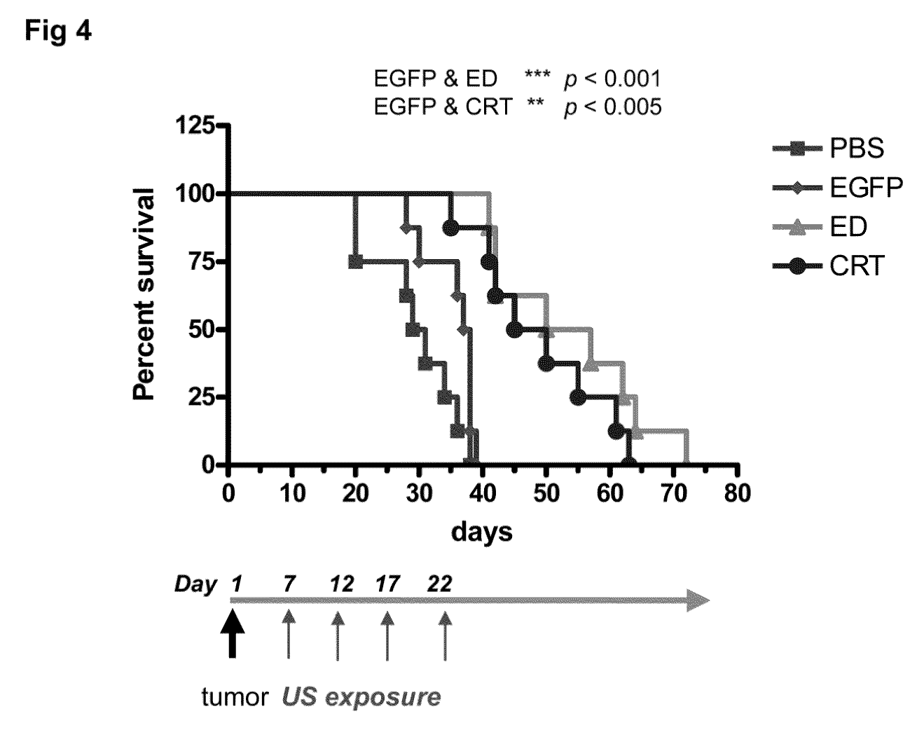

Similar findings were also found in mice with 7-day-old orthotopic liver tumor (i.e., tumors growing in the liver). Results were provided in FIG. 3. Ultrasound therapy given either intermittently (FIG. 3A) or consecutively (FIG. 3B), successfully reduced the tumor weight for at least 50%, as compared with that of the control animals. The survival curve for mice with orthotopic liver tumor receiving intermittent ultrasound therapy of ED or CRT was given in FIG. 4. Life expectancy increased for about 1.6˜1.8 times after mice being treated with ultrasound therapy. Life span for animals receiving ED and CRT treatment was prolonged to about 72 days and 63 days, respectively, as compared with 38˜40 days of the control animals (i.e., animals without any DNA treatment).

### Acoustically Delivery of ED or CRT Plasmid DNA Reduces Multifocal Liver Tumors in Rats

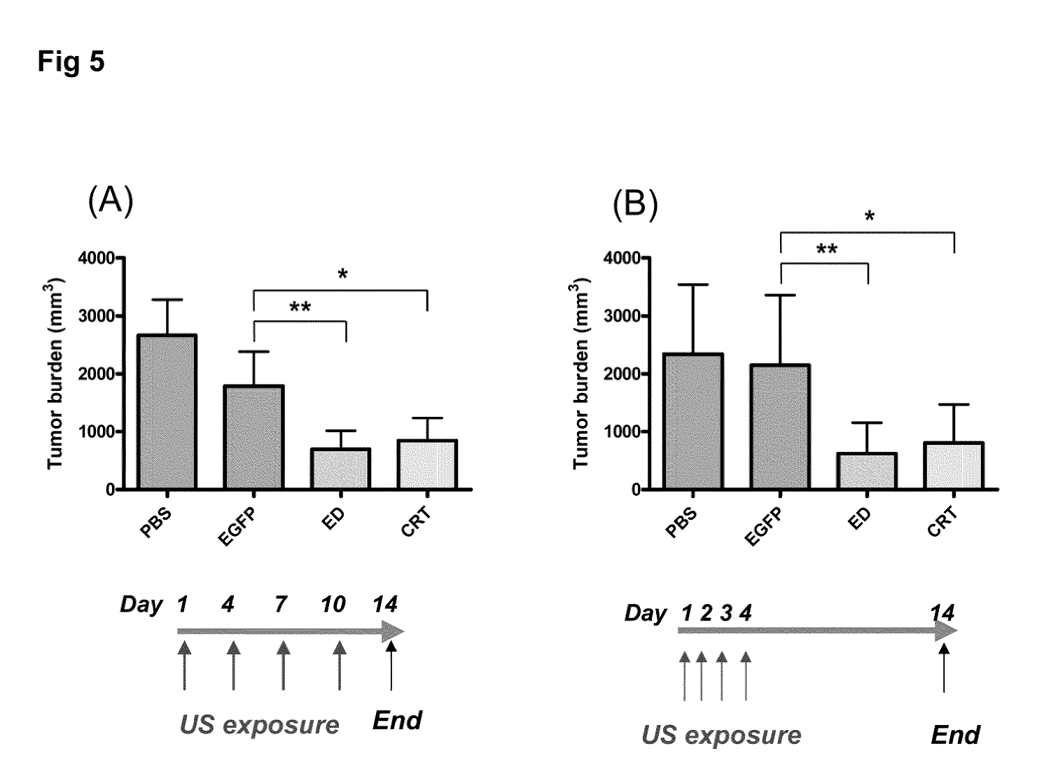

Primary liver tumors were induced in Wistar rats with DEN as described above. Animals were then given ultrasound therapy either intermittently or consecutively at indicated times, as illustrated in FIG. 5. It is clear from FIG. 5 that both ED and CRT plasmid DNA, administered either intermittently or consecutively, are effective in regressing the growth of the multifocal liver tumors, with tumor volume being successfully reduced to more than half of the control animals after acoustically delivery of the DNA solution for at least 4 times.

Combined Therapy with Ad/GM-CSF+Ad/IL-12 and Acoustically Delivery of ED- or CRT-Plasmid DNA Reduce Orthotopic Liver Tumors

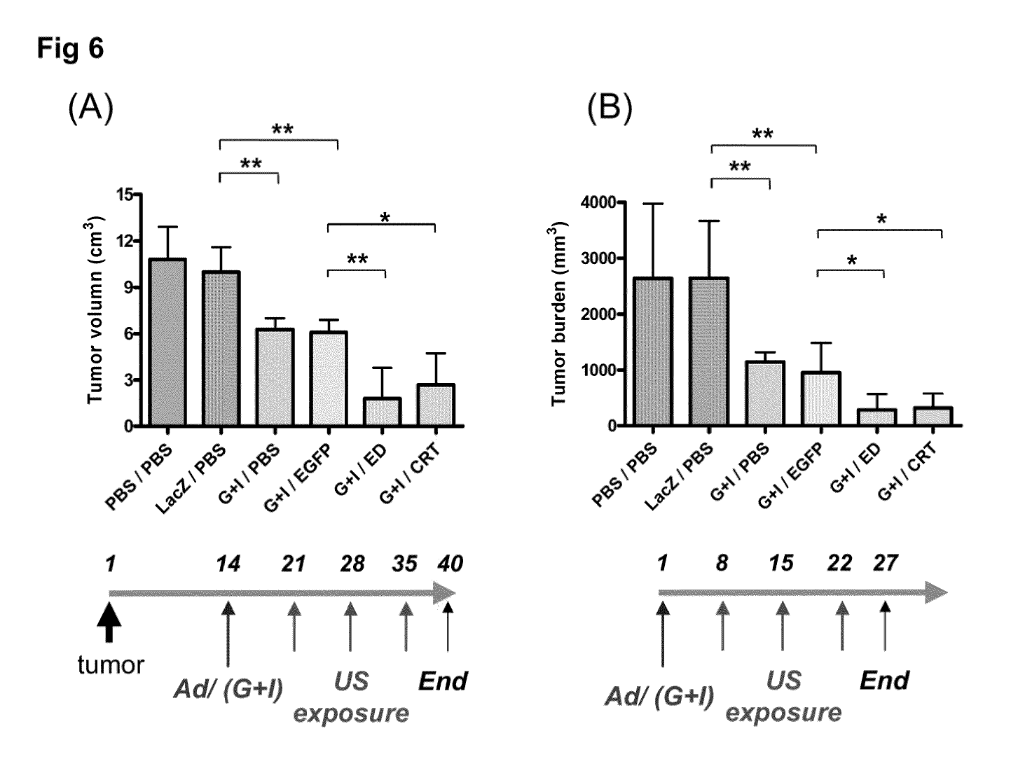

Mice were inoculated with liver tumor cells intrahepatically in accordance with the procedures described above. Mice with large tumor burden (i.e., 14-days old tumor) were used in this study. Each mouse received one shot of immunotherapy of Ad/GM-CSF+Ad/IL-12 (i.e., 0.5×10⁹ PFU/each) on day 14, followed by intermittent ultrasound ED- or CRT-plasmid DNA therapy on days 21, 28, and 35, respectively. Animals were then sacrificed on day 40, tumors were excised out, and their volumes were measured accordingly. Result was provided in FIG. 6(A). Immunotherapy with two adenoviral vectors (Ad/GM-CSF+Ad/IL-12) reduced the tumor volume to about 56˜58% of the control animals. Application of ultrasound ED or CRT plasmid DNA therapy shortly after adenoviral treatment further enhanced the reduction of the tumor volume for another 47% or 33%, respectively.

Alternatively, primary liver tumors were induced in Wistar rats with DEN as described above, then each rat received one shot of immunotherapy of Ad/GM-CSF+Ad/IL-12 (i.e., 0.5×10⁹ PFU/each) on day 1, followed by intermittent ultrasound ED- or CRT-plasmid DNA therapy on days 8, 15, and 22, respectively. Animals were then sacrificed on day 27, tumors were excised out, and their volumes were measured accordingly. Result was provided in FIG. 6(B). Immunotherapy with two adenoviral vectors (Ad/GM-CSF+Ad/IL-12) reduced the tumor volume to about 40% of the control animals. Application of ultrasound ED or CRT plasmid DNA therapy shortly after adenoviral treatment further enhanced the reduction of the tumor volume for another 29% or 28%, respectively.

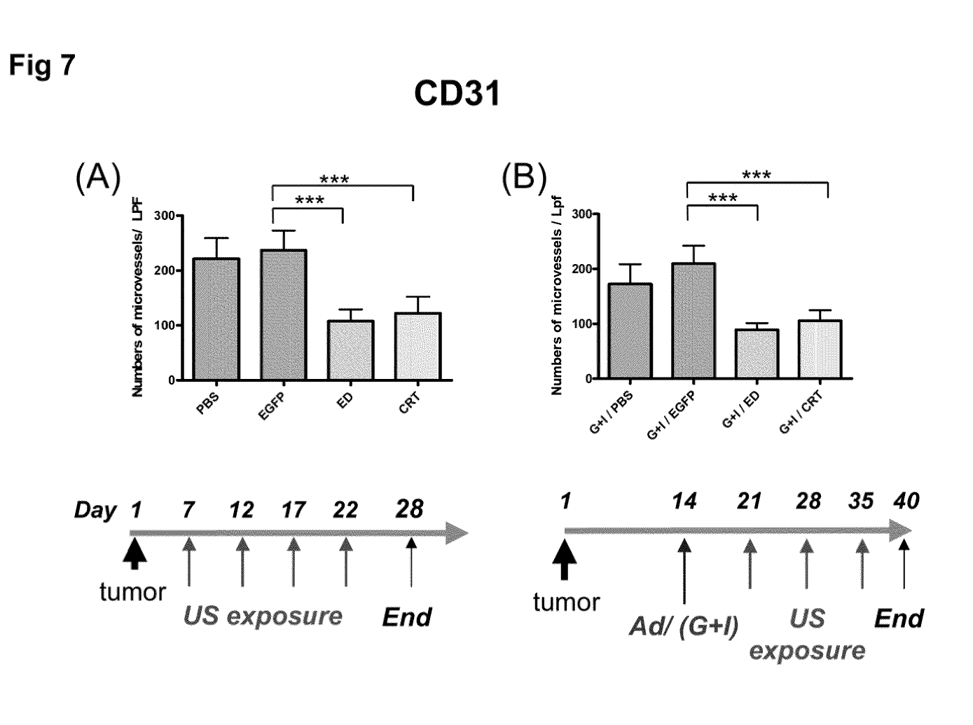

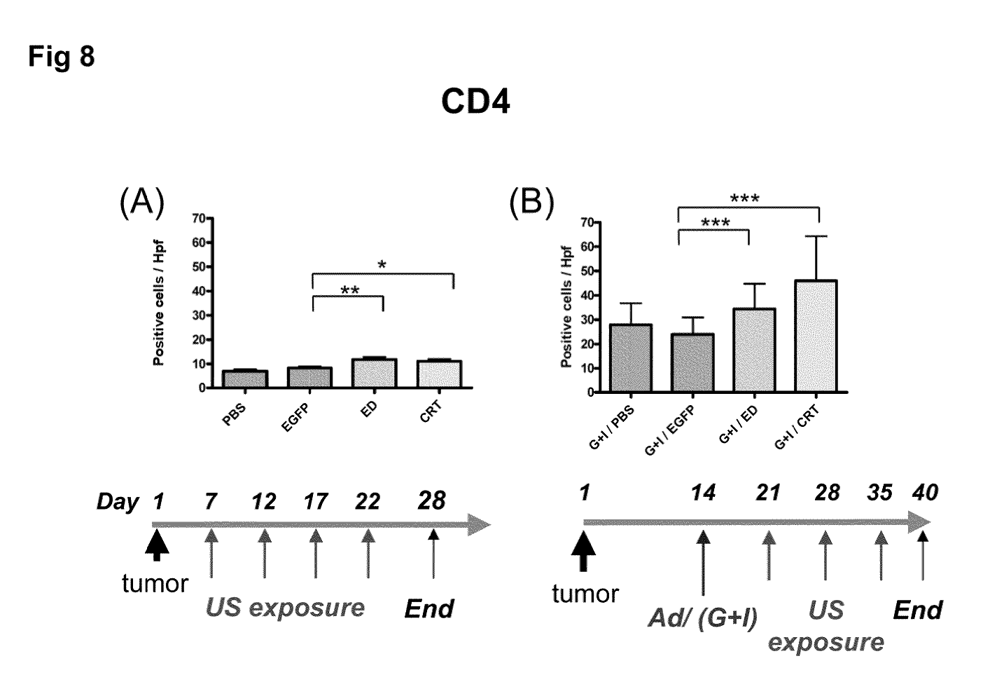

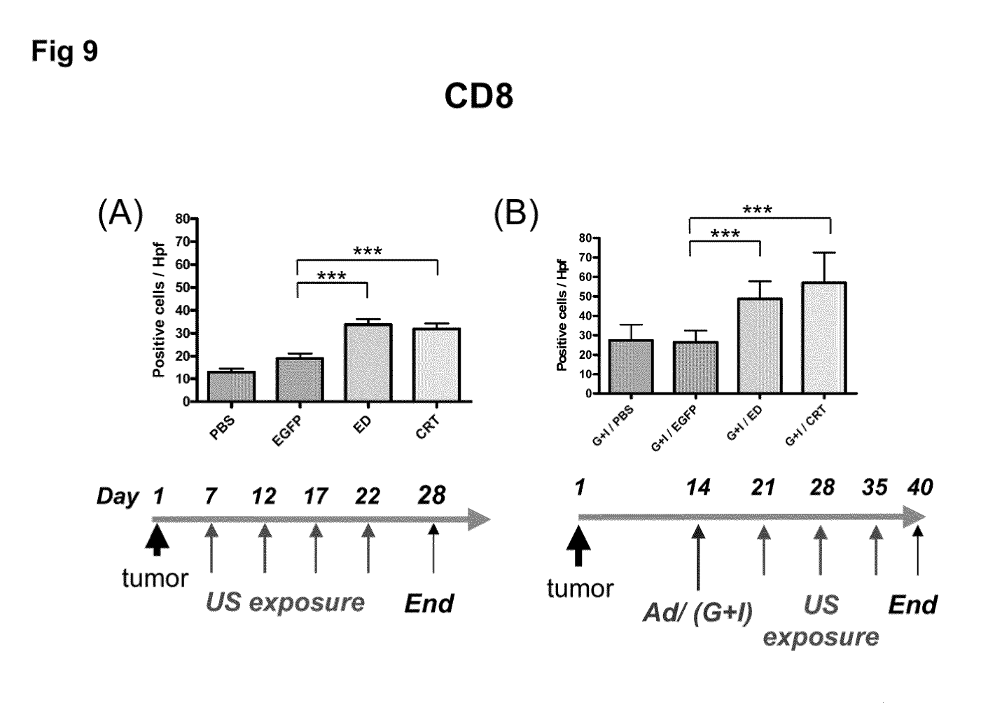

Immunohistochemistry staining confirms that the ultrasound therapy alone, as well as the combined therapy (i.e., combination of ultrasound ED- or CRT-plasmid DNA therapy and another two adenoviral vectors (Ad/GM-CSF+Ad/IL-12)) all significantly reduced the number of CD31+ cells found in the tumor (FIG. 7). Moreover, while CD4 positive cells and CD8 positive cells were greatly induced by immunotherapy with two adenoviral vectors (Ad/GM-CSF+Ad/IL-12), they were further increased significantly by the combined therapy (FIGS. 8 and 9).

The foregoing description of various embodiments of the disclosure has been presented for purpose of illustration and description. It is not intended to be exhaustive or to limit the disclosure to the precise embodiments disclosed. Numerous modifications or variations are possible in light of the above teachings. The embodiments discussed were chosen and described to provide the best illustration of the principles of the disclosure and its practical application to thereby enable one of ordinary skill in the art to utilize the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. A therapeutic composition for acoustically delivery to a subject for treating a tumor, comprising:
an effective amount of a plasmid nucleic acid suspended in a dispersed medium, and the plasmid nucleic acid encodes a polypeptide selected from the group consisting of endostatin (ED) and calreticulin (CRT); and
an effective amount of a microbubble contrast agent;
wherein the plasmid nucleic acids and the microbubble contrast agent are mixed in a ratio of 7:3 (v/v), and the therapeutic composition is capable of reducing a size of the tumor.

2. The composition of claim 1, wherein the microbubble contrast agent is composed of a shell and a gas core, wherein the shell is formed by a material selected from the group consisting of albumin, galactose, lipid, polymer and combinations thereof; and the gas core is formed by any of air, octafluoropropane, perfluorocarbon, sulfur hexafluoride or nitrogen.

3. The composition of claim 2, wherein the microbubble contrast agent has a lipid shell and a gas core formed by sulfur hexafluoride, and has an averaged diameter of about 2.5 μm.

4. The composition of claim 1, wherein the dispersed medium is any of water, a buffer solution, an isotonic sodium chloride solution, oils, or fatty acids.

5. The composition of claim 1, wherein the composition is delivered by the steps of:
parenterally administering from a site remote from a location of the tumor; and
exposing the parenterally administering site to ultrasound waves to enhance the delivery of the composition.

6. The composition of claim 5, wherein the parenterally administering step is intramuscularly injection.

7. The composition of claim 5, wherein the ultrasound waves have an intensity of about 0.5˜4 W/cm² and are administrated for a duration of about 1˜20 min

8. The composition of claim 5, wherein the ultrasound waves have an intensity of 2 W/cm² and are administrated for about 10 min.

9. The composition of claim 5, wherein the composition is administered every 1˜10 days with a dose of 10 μg˜10 mg plasmid nucleic acid/Kg body weight or 1˜100 mg polypeptide/Kg of body weight for at least 1˜10 times.

10. The composition of claim 9, wherein the composition is administered every 7 days with a dose of 10 μg˜10 mg plasmid nucleic acid/Kg body weight or 1˜100 mg polypeptide/Kg of body weight for at least 5 times

11. The composition of claim 9, wherein the composition is administered consequently every day with a dose of 10 μg˜10 mg plasmid nucleic acid/Kg body weight or 1˜100 mg polypeptide/Kg of body weight for at least 5 days.

12. The composition of claim 1, wherein the subject is a human.

13. The composition of claim 1, wherein the tumor is a malignant tumor selected from the group consisting of pancreatic cancer, lung cancer, colon cancer, gastric cancer, breast cancer, prostate cancer, hepatocellular carcinoma, melanoma, glioblastoma, brain tumor, hematopoietic malignancies, retinoblastoma, renal cell carcinoma, head and neck cancer, cervical cancer, esophageal cancer, and squamous cell carcinoma.

14. The composition of claim 13, wherein the tumor is a hepatic tumor.

15. The composition of claim 1, wherein the subject has received surgical treatment or radiotherapy for the tumor prior to receiving the composition.

16. The composition of claim 1, wherein the composition is administered to the subject before, at the same time or after a chemotherapeutic agent is administered.

17. The composition of claim 16, wherein the chemotherapeutic agent is selected from the group consisting of cisplatin, carboplatin, oxaliplatin, azathioprine, mercaptopurine, vincristine, vinblastine, vinorelbine, vindesine, paclitaxel, docetaxel, camptothecins, irinotecan, topotecan, amsacrine, etoposide, etoposide, phosphate, teniposide, dactinomycin, trastuzumab and cetuximab, rituximab.

18. The composition of claim 1, wherein the composition is administered before, at the same time or after administering an adenoviral vector of a nucleic acid encoding a polypeptide selecting from the group consisting of GM-CSF, IL-12, ED and PEDF.

19. The composition of claim 18, wherein the adenoviral vector is administered intratumorally to the subject before the composition is administered.

20. A method of acoustically delivering a therapeutic composition to a subject pre-diagnosed with a tumor, comprising:
(a) parenterally administering the therapeutic composition of claim 1 from a site remote from a tumor location; and
(b) exposing the parenterally administering site of step (a) to ultrasound waves to enhance the delivery of the therapeutic composition.

22. The method of claim 20, wherein the parenterally administering step is intramuscularly injection.

23. The method of claim 20, wherein the dispersed medium is any of water, a buffer solution, an isotonic sodium chloride solution, oils, or fatty acids.

23. The method of claim 20, wherein the microbubble contrast agent is composed of a shell and a gas core, wherein the shell is formed by a material selected from the group consisting of albumin, galactose, lipid, polymer and combinations thereof; and the gas core is formed by any of air, octafluoropropane, perfluorocarbon, sulfur hexafluoride or nitrogen.

24. The method of claim 20, wherein the ultrasound waves have an intensity of about 0.5˜4 W/cm² and are administered for about 1˜20 min.

25. The method of claim 24, wherein the ultrasound waves have an intensity of about 2 W/cm² and are administered for about 10 min.

26. The method of claim 20, wherein the method is repeated every 1˜10 days with a dose of 10 μg˜10 mg nucleic acid/Kg body weight or 1˜100 mg polypeptide/Kg of body weight for at least 1˜10 times.

27. The method of claim 26, wherein the method is repeated every 7 days with a dose of 10 μg˜10 mg nucleic acid/Kg body weight or 1˜100 mg polypeptide/Kg of body weight for at least 5 times.

28. The method of claim 26, wherein the method is repeated consequently every day with a dose of 10 μg˜10 mg nucleic acid/Kg body weight or 1˜100 mg polypeptide/Kg of body weight for at least 5 days.

29. The method of claim 20, wherein the subject is a human.

30. The method of claim 20, wherein the tumor is a malignant tumor selected from the group consisting of pancreatic cancer, lung cancer, colon cancer, gastric cancer, breast cancer, prostate cancer, hepatocellular carcinoma, melanoma, glioblastoma, brain tumor, hematopoietic malignancies, retinoblastoma, renal cell carcinoma, head and neck cancer, cervical cancer, esophageal cancer, and squamous cell carcinoma.

31. The method of claim 30, wherein the tumor is a hepatic tumor.

32. The method of claim 20, wherein the subject has received surgery or radiotherapy for the tumor prior to being subjected to the method.

33. The method of claim 20, wherein the method further comprises administering to the subject a chemotherapeutic agent before, at the same time or after initiating the method.

34. The method of claim 33, wherein the chemotherapeutic agent is selected from the group consisting of cisplatin, carboplatin, oxaliplatin, azathioprine, mercaptopurine, vincristine, vinblastine, vinorelbine, vindesine, paclitaxel, docetaxel, camptothecins, irinotecan, topotecan, amsacrine, etoposide, etoposide phosphate, teniposide, dactinomycin, trastuzumab and cetuximab, rituximab.

35. The method of claim 20, further comprises administering to the subject an adenoviral vector of a nucleic acid encoding a polypeptide selecting from the group consisting of granulocyte-macrophage colony-stimulating factor (GM-CSF), interleukin-12 (IL-12), endostatin (ED), and pigment epithelium-derived factor (PEDF), before, at the same time or after initiating the method.

36. The method of claim 35, wherein the adenoviral vector is administered intratumorally to the subject before initiating the method.
